(19) United States
(12) Patent Application Publication
(10) Pub. No.: US 2026/0137188 A1
(43) Pub. Date: May 21, 2026

(54) HAIRDRYER

(71) Applicant: TIME LINK CO., LTD, Suwon-si (KR)

(72) Inventor: Jae Hyun JUNG, Suwon-si (KR)

(73) Assignee: TIME LINK CO., LTD, Suwon-si (KR)

(21) Appl. No.: 19/121,868

(22) PCT Filed: Oct. 18, 2023
(86) PCT No.: PCT/KR2023/016195

§ 371 Date: Apr. 17, 2025

(30) Foreign Application Priority Data
Oct. 18, 2022 (KR) 10-2022-0133741

(51) Int. Cl. A45D 20/12; A61L 9/12

(52) U.S. Cl. CPC A45D 20/12; A61L 9/12; A61L 2209/15

(57) ABSTRACT

Disclosed is a hair dryer configured such that a portion of external air bypasses a heater and is discharged with a fragrance. The hair dryer disclosed herein comprises: a housing having an inflow path through which external air flows in, first and second blowing paths for blowing air flowed in from the inflow path, and an air discharge part for discharging external air; a heating element installed on the first blowing path to heat the air passing there-through; a handle installed on the outside of the housing; a blower part, installed in at least one of the inflow path of the housing and the inside of the handle, to draw in external air and inject the air to the first and second blowing paths; and a fragrance emitter mounting part which is formed in the housing to be in communication with the second blowing path and on which the fragrance emitter is removably mounted, so that the fragrance emitted from the fragrance emitter can bypass the heating element and be discharged through the air discharge part.

### TECHNICAL FIELD

The present invention relates to a hair dryer, and more particularly to a hair dryer in which a portion of external air is allowed to bypass the heater and be blown out with a fragrance.

### BACKGROUND ART

Generally, a hair dryer is used to dry wet hair or transform the current hairstyle into a desired one by blowing hot or room-temperature air.

A typical hair dryer draws in external air using a built-in motor and fan, and discharges hot or cold air by passing the air through a heating element that is either activated or not. Such devices may further comprise a temperature sensor for preventing overheating of the heating element or for controlling the temperature of the discharged air.

A known hair dryer includes a fragrance discharge function, such as the one described in Patent Document 1. Referring to FIG. 1, a hair dryer includes a body 1, a perfume reservoir 3 containing perfume mounted on the outlet side of the body 1, and a scent wand 5 connected to the perfume reservoir 3. The perfume contained in the perfume reservoir 3 may permeate the scent wand 5 and be exposed to the outside. Subsequently, the hair dryer may be operated to generate a blast of air, which may come into contact with the scent wand 5 and release the fragrance.

On the other hand, there is a problem with hot air blowing, where the fragrance reacts chemically with the hot air and changes its molecular structure, resulting in a disgusting odor, or the original smell is altered, causing the unwanted odor to be absorbed by the hair and offensive to the user or others.

### DISCLOSURE OF INVENTION

### Technical Problem

The present invention is revised to solve the above-mentioned problems, and aims to provide a hair dryer having a double ventilation path, wherein the heated air and the air containing the fragrance are ejected through separate discharge paths, so that the fragrance is prevented from deteriorating and the original smell of the fragrance is ejected.

### Solution to Problem

To achieve the above object, a hair dryer according to the present invention comprises: a housing having an inlet path through which external air is introduced, first and second blowing paths for blowing air introduced from the inlet path, and an air outlet for discharging the external air; a heating element disposed in at least one of the first and second blowing paths and configured to heat the air passing therethrough; a handle disposed on an exterior of the housing; a blowing unit disposed in at least one of the inlet path of the housing and an interior of the handle, the blowing unit configured to draw in external air and inject the air into at least one of the first and second blowing paths; and a scent mounting portion formed in the housing and in communication with at least one of the first and second blowing paths, the scent mounting portion configured to removably receive a scent emitter, wherein scent emitted from the scent emitter is discharged while bypassing the heating element.

The hair dryer may further comprise a filter installed in the housing and configured to prevent foreign substances from entering the interior of the housing.

The housing may further comprise a connecting passage connecting the inlet path and the second blowing path, and through which the external air flows in a direction opposite to the direction of air flow through the inlet path.

The housing may include: a first partition wall separating the inlet path from the connecting passage; a wind shield configured to divert air flowing through the inlet path into the first blowing path and the connecting passage; a second partition wall separating the first blowing path from the second blowing path; and a third partition wall separating the connecting passage from the second blowing path, wherein an opening/closing port penetrating the third partition wall is formed at a rear end of the third partition wall.

The scent mounting portion may further comprise an openable/closable portion configured to open the opening/closing port when the scent emitter is mounted and to close it when the scent emitter is removed.

The openable/closable portion may comprise: a resilient member accommodated and supported by a support step having an expanded inner diameter at the rear portion of the third partition wall; and a sliding member slidably installed by the pressing force of the scent emitter and a restoring force of the resilient member.

The openable/closable portion may further include a pressing pin formed on a portion of the sliding member facing the scent emitter and configured to penetrate the scent mounting portion, wherein when the scent emitter is mounted to the scent mounting portion, the pressing pin and sliding member are pushed, and the resilient member is compressed, thereby opening the opening/closing port, and when the scent emitter is removed, the sliding member returns to its original position by the restoring force of the resilient member, thereby closing the opening/closing port.

The air outlet may comprise a first air outlet and a second air outlet for respectively discharging the external air blown through the first and second blowing paths, and the scent mounting portion may be provided at the second air outlet.

The second blowing path may be positioned to surround an outer side of the first blowing path, thereby preventing the exterior of the housing from being heated by the air heated by the heating element.

The housing may be a cylindrical shape in which an inlet narrows toward one end facing the blowing unit, and may include a partition wall formed with an opening at a center of the one end that faces the blowing unit, and may further comprise a shutter configured to open and close the first blowing path in response to user operation.

The hair dryer may further comprise a scent emitter housing configured to receive the scent emitter, the scent emitter housing including a shutter that opens and closes in response to user operation.

An alarm unit configured to notify a user of whether the shutter is opened or closed may further be included.

The housing may further include a seating portion having an insertion hole formed through the housing, and the scent mounting portion may be removably mounted to the seating portion.

The scent mounting portion may further comprise a magnet configured to hold the scent emitter in place by magnetic force.

The scent mounting portion, which is a component of the hair dryer according to the present invention, may be installed through the insertion hole so as to be positioned in the second blowing path and may include: a guide portion configured to guide the emitted scent into the second blowing path; a mounting body having a working space formed on one side of the guide portion; a resilient member disposed in the working space; a sliding member slidably disposed in the working space and configured to move by a pressing force of the scent emitter and a restoring force of the resilient member; and a mounting cover installed on one side of the housing to cover the seating portion and formed with a working hole through which a portion of the sliding member protrudes, wherein the magnet is mounted on the mounting cover.

The hair dryer according to the present invention may further comprise a nozzle mounted on a front end of the housing, wherein the nozzle includes first and second discharge paths formed by a dividing wall extending from the boundary between the first and second blowing paths.

### Advantageous Effects of Invention

The hair dryer according to the present invention includes a first blowing path through which a portion of the external air passes and is discharged after being heated by the heating element, and a second blowing path through which the remaining external air bypasses the heating element and is discharged at room temperature after passing the scent emitter.

This configuration fundamentally prevents deterioration of scent quality due to heated air and allows the original scent to be transferred to the user's hair.

Further, the hair dryer according to the present invention enables air to be discharged from both the first and second blowing paths when the scent emitter is mounted, and to be discharged only from the first blowing path when the scent emitter is not mounted, due to the openable/closable portion provided in the second blowing path.

The filter provided in the scent mounting portion prevents foreign substances from entering the main body of the hair dryer when the scent emitter is mounted. By including a nozzle with first and second discharge paths, the hair dryer allows air heated through the first blowing path and air containing scent to be separately discharged without mixing inside or outside the housing.

Since the first blowing path including the heating element is located on the inner side of the housing, and the second blowing path through which unheated air flows is located to surround the first blowing path, the external surface of the housing can be prevented from heating due to the heated air.

Furthermore, the hair dryer according to the present invention includes a configuration in which the scent emitter is mounted to the scent mounting portion provided at the second air outlet of the housing. This prevents residual scent from the scent emitter from lingering inside the housing. Accordingly, there is no need to clean the housing to remove any residual scent.

### BRIEF DESCRIPTION OF DRAWINGS

FIG. 1 is a perspective view of a conventional hair dryer.

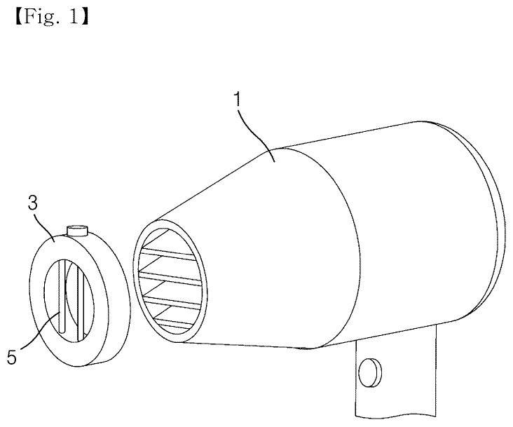

FIG. 2 is a perspective view of a hair dryer according to a first embodiment of the present invention.

FIG. 3 is a sectional view taken along line III-III of FIG. 2.

FIG. 4 is an enlarged sectional view of a portion showing the communication between a connecting passage and a second airflow path in FIG. 2.

FIG. 5 is a perspective view of a key component separated from the hair dryer shown in FIG. 2.

FIG. 6 is a partial perspective view of a hair dryer according to a second embodiment.

FIG. 7 is a sectional view of the hair dryer shown in FIG. 6.

FIG. 8 is a perspective view of the scent mounting portion of FIG. 6.

FIG. 9 is a sectional view of a variant of the embodiment in FIG. 6.

FIG. 10 is a schematic sectional view of a hair dryer according to a third embodiment.

FIG. 11 is a schematic sectional view of a variation of the third embodiment.

FIG. 12 is another schematic sectional view of a variation of the third embodiment.

FIG. 13 is a schematic sectional view of a hair dryer according to a fourth embodiment.

FIG. 14 is a schematic sectional view of a hair dryer according to a fifth embodiment.

FIGS. 15A and 15B illustrate the scent mounting portion of FIG. 14, showing the shutter in an open and closed state, respectively.

### MODE FOR THE INVENTION

Hereinafter, with reference to the accompanying drawings, embodiments will be described in detail to facilitate the practice of the present invention by one having ordinary skill in the art to which it pertains. Furthermore, among the configurations mentioned in the various embodiments, mutually similar or related configurations may be substituted, replaced, or added between embodiments. However, in order to describe the principles of operation of the embodiments of the present invention in detail, specific descriptions of well-known features or configurations are omitted if such descriptions would unnecessarily obscure the essence of the present invention.

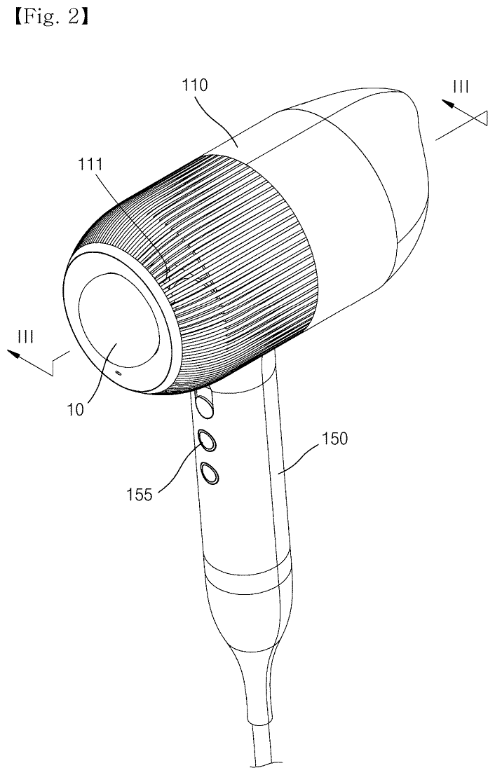

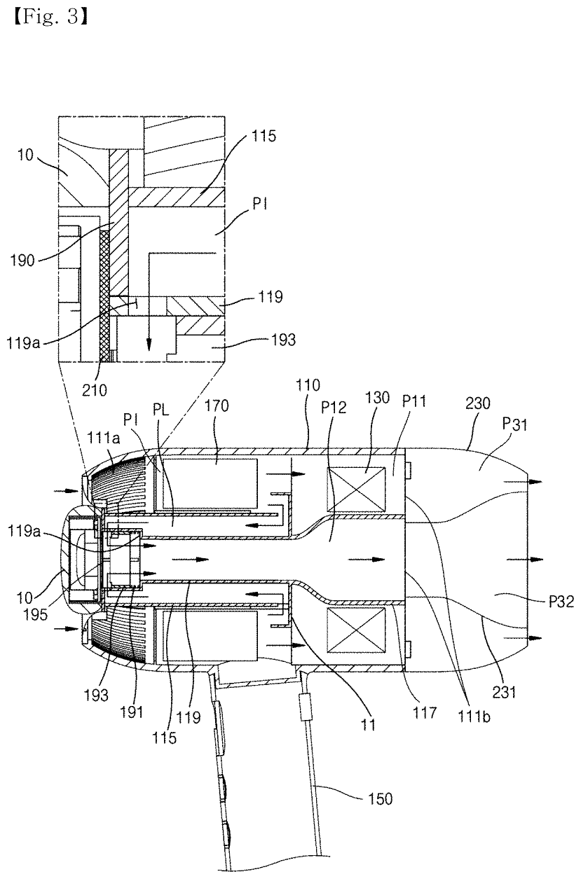

FIG. 2 is a perspective view of a hair dryer according to a first embodiment of the present invention, and FIG. 3 is a sectional view III-III of FIG. 2.

Referring to FIGS. 2 and 3, a hair dryer according to the first embodiment of the present invention may include a housing 110, a heating element 130, a handle 150, a blowing unit 170, and a scent mounting portion 190.

The housing 110 is formed with an inlet path PI in which external air flows in, first and second blowing paths P11 and P12 for blowing the air flowing in from the inlet path PI, and an air outlet 111b for discharging the external air. Here, the inlet path PI is formed as a single inlet path, and the air entering the inlet path can be branched and blown to the first blowing path P11 and the second blowing path P12. It is also possible that an inlet path is formed corresponding to each blowing path.

The housing 110 may further include a connecting passage PL that connects the inlet path PI and the second blowing path P12. To this end, the housing 110 may include a first to third partition walls 115, 117 and 119, and a wind shield 113 provided in the internal space. The second blowing path P12 may be to around the first blowing path P11, as shown in FIG. 2. The first and second blowing paths P11 and P12 may be formed adjacent to each other.

The housing 110 may further include an air inlet 111a formed at the rear end of the inlet path PI, allowing external air to be introduced to the interior. The air inlet 111a may be provided at the rear of the housing 110 and may comprise a plurality of comb-like slots or holes to allow external air to enter the interior. The air inlet portion 111a may also be at the rear of the housing 110 and may be machined around the periphery of the scent emitter 10.

The heating element 130 is installed in the first blowing path P11 to heat the air passing through the first blowing path P11.

The handle 150 is used for holding the hair dryer and may extend approximately downward from the housing 110. The handle 150 may include a control unit 155 configured to control operation of the hair dryer. The control unit 155 may be mounted on the handle 150 and allow a user to select whether to operate the blowing unit 170 and adjust its intensity, as well as to enable heating by the heating element 130 and adjust the temperature. The control unit 155 may be electrically connected to the power supply for the blowing unit 170 and the heating element 130.

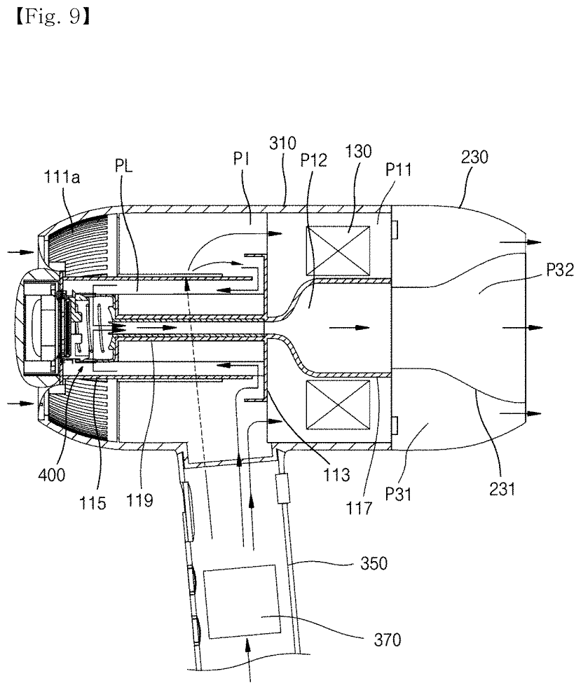

The blowing unit 170 may be installed in the inlet path PI of the housing 110, as shown in FIG. 3. The blowing unit 170 may include a blowing fan for drawing in external air and a power source for operating the blowing fan. The blowing unit 170 may generate suction to introduce air through the air inlet 111a and move it along the inlet path PI, the connecting passage PL, and the first and second blowing paths P11 and P12. The blowing unit 170 may also be mounted in the handle 150 as shown in FIG. 9, which will be described later.

The scent mounting portion 190, where the scent emitter 10 is removably mounted, may be formed in the housing 110 to be in communication with the second blowing path P12, i.e., the scent mounting portion 190 may be provided at the rear of the second blowing path P12. FIG. 3 illustrates an example where the scent emitter 10 is magnetically attached to the housing 110, e.g., with a permanent magnet embedded in the emitter and a magnetic material in the mounting portion, or vice versa. Alternatively, the scent mounting portion 190 may be provided with a permanent magnet and the scent emitter 10 may comprise a magnetic material. By configuring the scent emitter 10 in this manner, a user can easily attach and detach the scent emitter 10 to the housing 110. In addition, the scent mounting portion 190 and the scent emitter 10 may be processed to be coupled by any one of a variety of couplings, including screw coupling, push fit coupling, or hook coupling, in addition to the magnetic coupling described above.

The hair dryer according to this embodiment may further include a filter 210 and a nozzle 230. The filter 210 is installed in the scent mounting portion 190 to prevent foreign substances from entering the housing 110, i.e., the filter 210 block the foreign substances from entering with the scent when scent is desired to be introduced from the scent emitter 10.

The nozzle 230 may be detachably mounted on the leading edge, i.e. the front part, of the housing 110. The nozzle 230 includes a dividing wall 231 extending from the boundary between the first blowing path P11 and the second blowing path P12. The nozzle 230 may have first and second discharge paths P31 and P32 separated by the separation wall 231.

The nozzle 230 may be constructed so that air traveling through the first blowing path P1 is concentrated and blown out, and the air traveling through the second blowing path P12 is diffused and blown out. Specifically, the nozzle 230 may have a structure in which two cylinders of roughly different diameters are superimposed, such that a first discharge path P31 is formed on the outside of the separating wall 231 and a second discharge path P32 is formed on the inside of the separating wall 231. Also, referring to FIG. 3, the front portion of the first discharge path P31 may be formed in a shape that narrows toward the end, and the front portion of the second discharge path P32 may be formed in a shape that widens toward the end. Accordingly, the fragrance moved through the second blowing path P12 may be discharged by diffusion from the nozzle 230, thereby spreading the fragrance throughout the user's hair.

In the following, the structure of the internal space of the housing 110 will be examined in detail.

External air enters the housing 110 through the air inlet 111a and is transported along the inlet path PI. The external air entering through the inlet path PI branches into at least two paths, i.e., a portion of the external air is transported through the first blowing path P11 where the heating element 130 is installed. The other part of the introduced air passes through the connecting passage PL and then through the second blowing path P12 and is transported to the outside of the housing 110 in a mixed state with the scent emitted from the scent emitter 10. The connecting passage PL connects the inlet path and the second blowing path, and guides the outside air to move in a direction opposite to the direction of movement of the outside air traveling through the inlet path. For this purpose, the housing 110 includes a first partition wall 115, a wind shield 113, a second partition wall 117, and a third partition wall 119 provided in the interior space of the housing 110.

The first partition wall 115, which separates the inlet path PI from the connecting passage PL, may be approximately cylindrical in shape, fixed at one end to a rear inner surface of the housing 110, and positioned near a wind shield 113, which is disposed at a front end and has a leading edge disposed in a middle portion of the housing 110. The wind shield 113 is provided at the air discharge end of the inlet path PI, and divides the air entering through the inlet path PI into the first blowing path P11 and the connecting passage PL. In other words, the wind shield 113 is deflected toward the housing 110 from the boundary of the second partition wall 117 and the third partition wall 119, is positioned between the blowing unit 170 and the heating element 130, and may be formed to partially encroach on the boundary of the inlet path PI and the first blowing path P11. The wind shield 113 may be deflected at its end towards the inlet path PI. Thus, a portion of the air traveling along the inlet path PI may be deflected by the wind shield 113 and enter the connecting passage PL.

The second partition wall 117 separates the first blowing path P11 from the second blowing path P12, and the third partition wall 119 separates the connecting passage PL from the second blowing path P12. Each of the second and third partition walls 117 and 119 may be substantially cylindrical in shape and may comprise interconnected ends, i.e., the second partition wall 117 and the third partition wall 119 may be integrally formed. At the rear end of the third partition wall 119, an opening/closing port 119a is formed that passes between the connecting passage PL and the second blowing path P12. Through the opening/closing port 119a, air flowing through the connecting passage PL can enter the second blowing path P12 on the inside of the third partition wall 119. The third partition wall 119 may be to provide a support step 119a with an expanded inner diameter over a portion of the rear portion of the scent emitter 10, and scent mounting portion 190 may be on the expanded portion. Additionally, a nozzle 230 may be removably mounted to the leading edge of the housing 110 and the leading edge of the second partition wall 117.

The scent mounting portion 190 may further include an openable/closable portion 191 that allows the opening/closing port 119a to open when the scent emitter is mounted and the opening/closing port 119a to close when the scent emitter is removed. The openable/closable portion 191 may include a resilient member 192 and a sliding member 193. The resilient member 192 is adapted to be on a support step 119a having an inner diameter extending from a rear portion of the third partition wall 119 for opening and closing the opening/closing port 119a. Each of the resilient member 192 and the sliding member 193 may be formed in a hollow shape so as not to obstruct the flow of air introduced through the opening/closing port 119a. The resilient member 510 may be, for example, a compressed coil spring. The sliding member 193 may be formed in a cylindrical shape and is slidably installed by the force of the scent emitter 10 and the restoring force the resilient member.

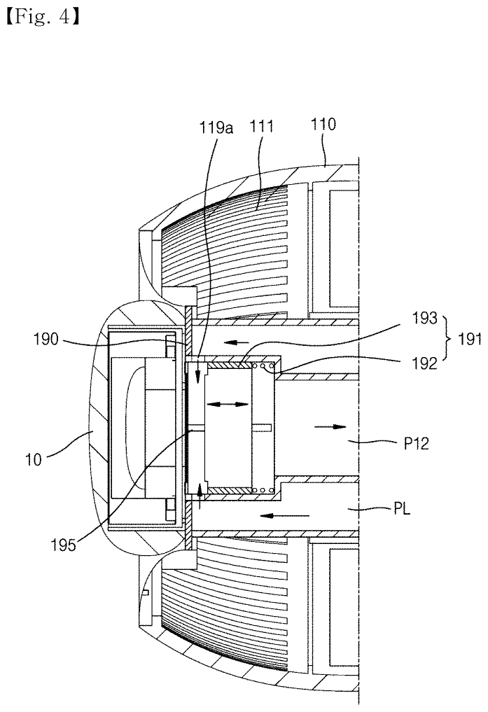

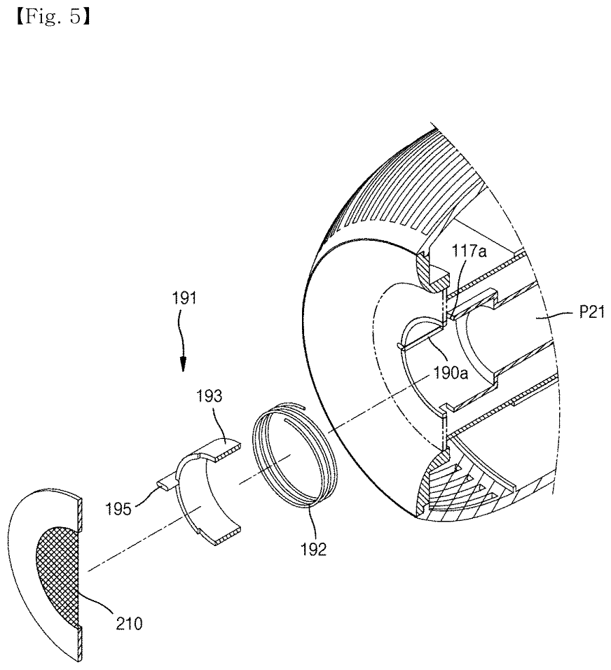

The openable/closable portion 191 may further comprise pressing pin 195 at one end on the scent side of the sliding member 193, positioned toward the scent mounting portion 190, as shown in FIGS. 4 and 5. Thus, when the scent emitter 10 is mounted on the scent mounting portion 190, pressing the pressing pin 195 causes the sliding member 193 to slide, thereby opening the opening/closing port 119a. At this time, the resilient member 192 is compressed. On the other hand, when the scent emitter 10 is removed, the sliding member 193 returns due to the elastic restoring force of the resilient member 192, and the opening/closing port 119a closes. Therefore, by closing the opening/closing port 119a when the scent emitter 10 is not in use, the external air entering the housing 110 can be moved only through the first blowing path P11, thereby preventing a decrease in the flow rate.

The pressing pin 195 fits into a guide 190a machined into the surface of the third partition wall 119, and can be slidably along the guide groove 190a.

The scent emitter 10 may be removably mounted to a scent mounting portion 190 at the rear of the housing 110. The scent emitter 10 may contain a scent substance, including a perfume, inside and may be partially open and exposed to the outside via a scent member including a scent test paper. When the scent emitter 10 is mounted on the scent mounting portion 190, the opening/closing port 119a is opened to allow external air to move through the second blowing path P12, and the scent emitted from the scent emitter 10 is mixed with the air on the second blowing path P12.

As described above, the hair dryer according to the first embodiment of the present invention allows the air heated by the heating element 130 and the at room temperature to flow through separate passages, thereby preventing the fragrance from becoming foul or pungent due to contact with warm air and ensuring a soft and pleasant scent.

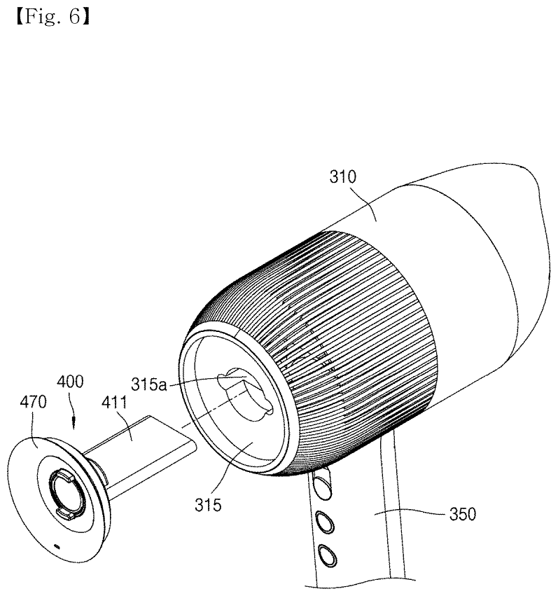

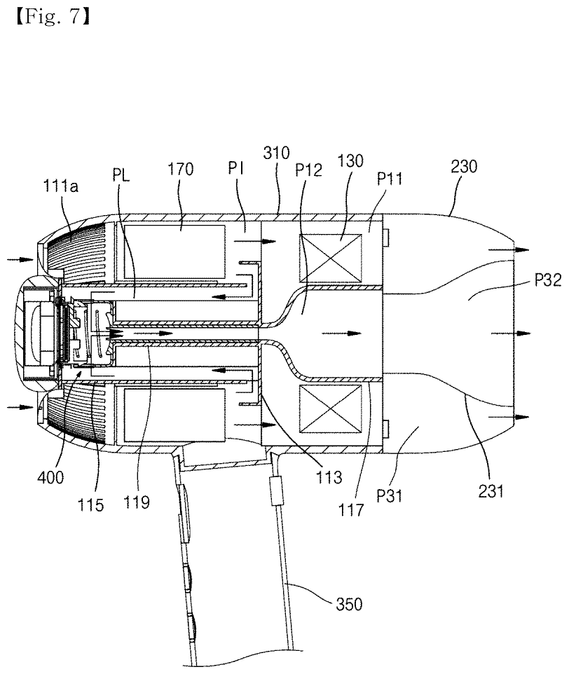

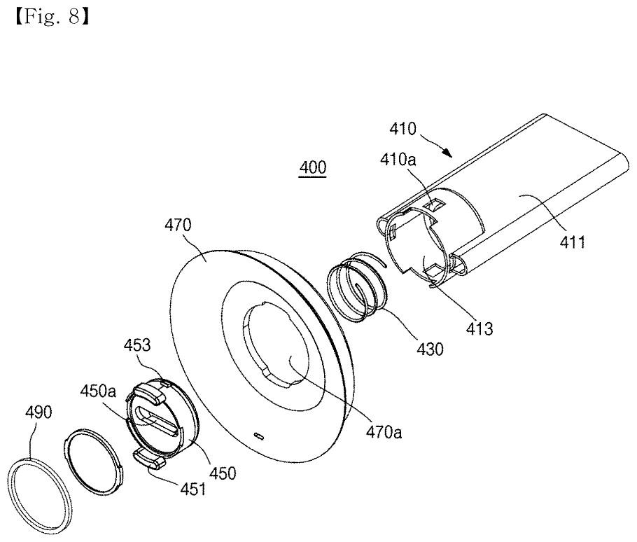

FIG. 6 is a partially isolated perspective view of a hair dryer according to a second embodiment of the present invention, FIG. 7 is a cross-sectional view of FIG. 6, and FIG. 8 is a partially isolated perspective view of the scent mounting portion of FIG. 6.

Referring now to FIGS. 6 through 8, a hair dryer according to a second embodiment of the present invention may include a housing 310, a heating element 130, a handle 350, a blowing unit 170, and a scent mounting portion 400. The housing 310 has an inlet path PI through which external air is introduced, first and second blowing paths P11 and P12 that branch from the inlet path PI, and an air outlet 111b through which external air is discharged. The housing 110 may further include a connecting passage PL connecting the inlet path PI and the second blowing path P12. To this end, the housing 110 may include a first to third partition wall 115, 117 and 119, and a wind shield 113 provided in the internal space.

The hair dryer according to this second embodiment differs from the hair dryer according to the first embodiment in that it includes a seating portion 315 with respect to the housing 310, and a scent mounting portion 400 is configured separately from the housing 310, but the other configurations are substantially the same. Therefore, the same drawing numbers are assigned to the substantially identical components, and a detailed description will be omitted, focusing on the scent mounting portion 400.

A seating portion 315 is installed at the rear of the housing 310. The seating portion 315 is formed with an insertion hole 315a that penetrates into and through the housing 310. The scent mounting portion 400 is removably mounted to the seating portion 315.

Referring to FIGS. 7 and 8, the scent mounting portion 400 may include a mounting body 410, a resilient member 430, a sliding member 450, and a mounting cover 470. The mounting body 410 is installed through the insertion hole 315a such that it is positioned within the second blowing path P12.

The mounting body 410 may include a guide portion 411 for guiding the emitted scent to move through the second blowing path P12, and a working space 413 formed on one side of the guide portion 411. The resilient member 430 is installed in the working space 413, and the sliding member 450 is installed in the working space 413, slidably by the force of the scent emitter 10 and the restoring force the resilient member 430. A mounting cover 470 is installed on one side of the housing 310 to cover the seating portion 315. The mounting cover 470 may have a working aperture 470a formed therein through which a portion of the sliding member 450 may be installed.

The sliding member 450 may further pressing pin 451 formed on one side of the scent emitter 10. Thus, when the scent emitter 10 is mounted to the scent mounting portion 400, pressing the pressing pin 451 causes the sliding member 450 to slide into the working space 413, compressing the resilient member 430 and opening the mounting 410a formed in the mounting body 410. The mounting bore 410a may be slidably and insertably coupled to a fixing member 453 installed on the sliding member 450. Here, the fixing member 453 may be formed as a hook structure and may be mutually coupled in a state of relative elastic deformation between the fixing member 453 and the mounting hole 410a.

Furthermore, a scent passage hole 450a may be formed in the center of the sliding member 450 through which the scent emitted from the scent emitter 10 is communicated. Depending on the relative position of the sliding member 450 with respect to the working space 413 with or without the scent emitter 10, the scent passage hole 450a may have a retractable configuration.

The scent mounting portion 400 is mounted to the mounting cover 470 and may further include a magnet 490 that magnetically secures the scent emitter 10 to the mounting cover 470.

FIG. 9 is a cross-sectional view of a variation of FIG. 6. Referring to FIG. 9, the internal space of the handle 350 may be used as a separate air passage, and a blowing unit 370 may be provided therein. In this case, an air inlet may be installed in the handle 350 so that outside air from the air inlet may be supplied to the inlet path PI, the connecting passage PL, the first and second blowing paths P11 and P12, which are formed in the housing 310 via the internal passage of the handle 350.

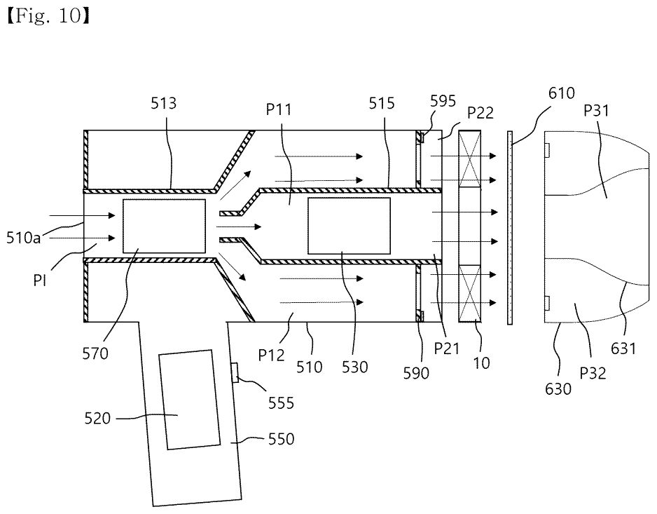

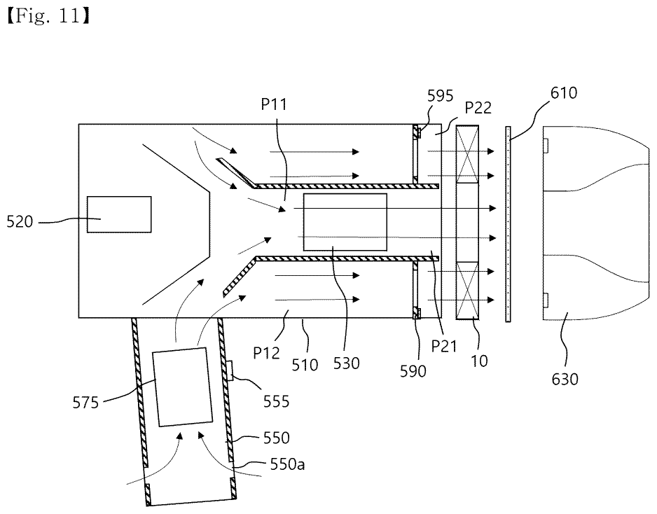

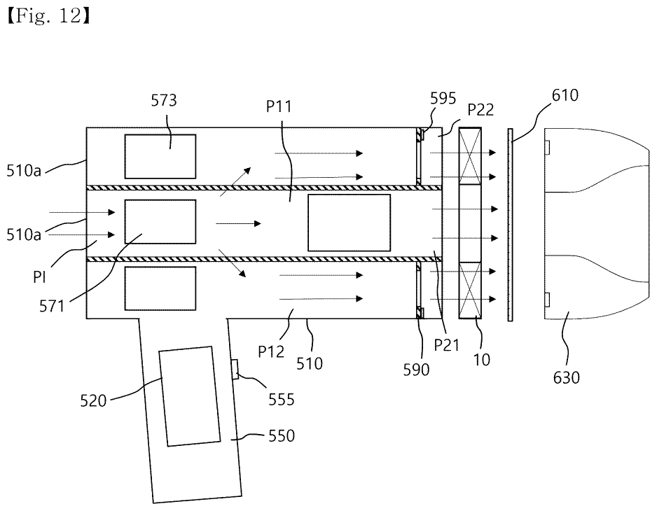

FIG. 10 is a cross-sectional view of a hair dryer according to a third embodiment of the present invention, and FIGS. 11 and 12 are variations of FIG. 10.

Referring to FIG. 10, a hair dryer according to a third embodiment of the present invention may include a housing 510, a heating element 530, a handle 550, a blowing unit 570, and a scent mounting portion 590.

The housing 510 is formed with an inlet path PI through which external air is introduced, first and second blowing paths P11 and P12 that branch from the inlet path PI, and first and second air outlets P21 and P22 through which air is discharged. The first air outlet P21 is where the air transported through the first blowing path P11 is discharged, and the second air outlet P22 is where the air transported through the second blowing path P12 is discharged. Thus, to form the inlet path PI, the first and second blowing paths P11 and P12, and the first and second air outlets P21 and P22, the housing 510 may include a partition wall 515 provided in the internal space. The first and second blowing paths P11 and P12 are separated by the partition wall 515, which may be formed as a cylindrical shape with open sides.

The housing 510 may further include an air inlet portion 510a formed at a rear end of the inlet path PI to allow external air to be introduced. The air inlet 510a may be provided at the rear of the housing 510 and may be shaped as a plurality of comb-like slots or a plurality of holes to allow external air to enter the housing 510.

The heating element 530 is installed in the first blowing path P11 to heat the air passing through the first blowing path P11. Here, the second blowing path P12 may be positioned to wrap around the outer side of the first blowing path P11. In other words, the first blowing path P11 in which the heating element 530 is installed is located on the inner side of the housing 510, and the second blowing path P12 through which the unheated air passes is located so as to wrap around the outer side of the first blowing path P11. Thus, the hair dryer according to the third embodiment of the present invention can prevent the outside of the housing from being heated by the air heated by the heating element 530.

The handle 550 is the portion of the hair dryer that is when in use, and may be to approximately from the housing 510. The handle 550 may include a control unit 555 for operating the hair dryer. The control unit 555 may be mounted on the handle 550 and may be configured to select whether the blowing unit 570 is operated and the blowing intensity, and to select whether the heating element 530 is heated and the heating temperature. Of course, the control unit 555 may be electrically connected to the power source of the blowing unit 570 and the heating element 530.

The blowing unit 570 may be in the inlet path PI of the housing 510, as shown in FIG. 10. The blowing unit 570 may comprise a blowing fan capable of drawing in external air and a power source capable of operating the blowing fan. The blowing unit 570 provides a suction force to inhale the external air, such that the external air is inhaled through the air inlet portion 510a and the inhaled air is moved along the inlet path PI, the first and second blowing paths P11 and P12.

The blowing unit may also be installed on the inside of the handle 550, as shown in FIG. 11. Referring to FIG. 11, an air inlet 550a is formed in the handle 550, and air introduced through the air inlet 550a is transported to the first and second blowing paths P11 and P12 by operation of the blowing unit 575. In this case, the inlet path PI and the partition wall can be changed to suit the installation position of the blowing unit 575.

Furthermore, as shown in FIG. 12, a plurality of blowing units may be provided and in each of the first blowing path P11 and the second blowing path P12. Referring to FIG. 12, the blowing unit includes a first blowing unit 571 and a second blowing unit 573. The first blowing unit 571 is installed in the first blowing path P11, and external air entering through the air inlet 510a is transported to the first blowing path P11. The second blowing unit 573 is installed in the second blowing path P12 so that the external air entering through the air inlet 510a is transferred to the second blowing path P12.

Here, the blowing units 570 and 175, the first and second blowing units 571 and 573, and the heating element 530 are controllable by a controller 520 provided in the housing 510. The controller 520 drives and controls the blowing unit and the heating element in response to a control signal applied by the operation part 555.

Referring now to FIG. 10, a scent mounting portion 590 may be formed in the housing 510 where the scent emitter 10 is removably mounted, in communication with the output end of the second blowing path P12. In other words, the scent mounting portion 590 may be provided at the second air outlet P22. FIG. 10 illustrates an example in which the scent emitter 10 is coupled to the scent mounting portion 590 by magnetic force, i.e., a permanent magnet 595 is provided on the scent mounting portion 590, and the scent emitter 10 side may be composed of a magnetic material. Alternatively, the scent emitter 10 may be provided with a permanent magnet and the scent mounting portion 590 may comprise a magnetic material. By configuring the scent emitter 10 in this manner, a user can easily attach and detach the scent emitter 10 to the housing 510. In addition, the scent mounting portion 590 and the scent emitter 10 may be processed to be coupled by any one of a variety of couplings, including screw couplings, push-pull couplings, or hook couplings, in addition to the magnetic couplings described above.

As described above the hair dryer according to a third embodiment of the present invention comprises a first blowing path P11 in which portion of the introduced external air is blown the heating element 530 and out, a second blowing path P12 in which the remaining external air is diverted and blown out at room temperature through the scent emitter 10, thereby essentially preventing the scent from being degraded by warm air and allowing the original scent to be applied to the hair.

Furthermore, the hair dryer according to a third embodiment of the present invention can prevent the scent emitter 10 from passing through the housing 510, thereby preventing residual scent from being distributed within the housing. This has the advantage of eliminating the need to clean the housing to remove lingering fragrance.

The hair dryer according to a third embodiment of the present invention may further comprise a filter 610. The filter 610 may be installed at the air outlet of the housing 510 to prevent debris contained in the air that is drawn in with the external air from being directed onto the user's hair.

The hair dryer according to the third embodiment of the present invention may further comprise a nozzle 630. The nozzle 630 may be removably mounted to a leading edge, or front portion, of the housing 510. The nozzle 630 includes a separator wall 631 extending from a boundary between the first blowing path P11 and the second blowing path P12. The nozzle 630 may have first and second discharge paths P31 and P32, respectively, separated by the separation wall 631.

The nozzle 630 may be configured such that the air traveling through the first blowing path P11 is concentrated and blown out, and the air traveling through the second blowing path P12 is diffused and blown out. Specifically, the nozzle 630 may have a structure in which two cylinders of roughly different diameters are superimposed, such that a first discharge path P31 is formed on the inside of the separation wall 631 and a second discharge path P32 is formed on the outside of the separation wall 631. Further, the front portion of the first discharge path P31 may be formed in a shape that narrows toward the end, and the front portion of the second discharge path P32 may be formed in a shape that widens toward the end.

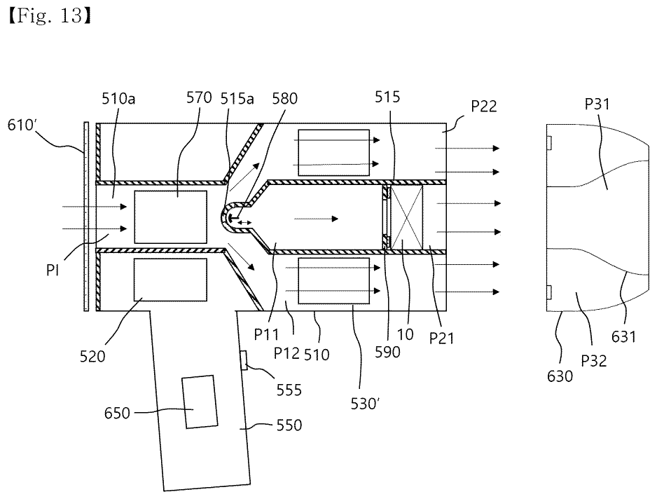

FIG. 13 is a schematic cross-sectional view of a hair dryer according to a fourth embodiment of the present invention.

Referring to FIG. 13, a hair dryer according to a fourth embodiment of the present invention may include a housing 510, a heating element 530′, a handle 550, and a blowing unit 570. Compared to the hair dryer according to the third embodiment of the present invention, it is distinguished by a change in the mounting position of the heating element 530′, the scent emitter 10 and the filter 610′, and by the opening and closing of the first blowing path P11, while the other parts are substantially the same and will not be described again.

The heating element 530′ may be installed within the second blowing path P12 to heat the air passing through the second blowing path P12.

The scent emitter 10 may be installed in an internal space of the housing 510 that forms a first blowing path P11, which is defined by a partition wall 515. The scent emitter 10 may further comprise a scent mounting portion 590 for mounting the scent emitter 10.

The partition wall 515 may be formed in a cylindrical shape, and a first portion of the partition wall 515 facing the blowing unit 570 may be formed in a shape with an opening that narrows toward the end, and an opening 515a may be formed in the center thereof.

The hair dryer according to the fourth embodiment may comprise a shutter 581 that opens and closes the first blowing path P11 in response to a user operation, i.e., the shutter 581 opens and closes the opening 515a. The shutter 581 may be configured to open and close the opening 515a in the manner shown in FIG. 13, or in another manner aligned with the direction of the blowing path.

As described above, the hair dryer according to the fourth embodiment of the present invention is capable of activating the function by opening and closing the first blowing path P11 in which the scent emitter 10 is installed at the option of the user. Further, by closing the shutter 581 to block the first blowing path P11 when the hair dryer is not in use, the scent accommodated in the scent emitter 10 can be minimized to be consumed when not in use.

Here, the hair dryer according to a fourth embodiment of the invention may further comprise an alarm unit 650 that can notify a user whether the scent function is activated, i.e., whether the shutter 581 is open or closed. The alarm unit 650 may include a display to provide visual information and/or a microphone to provide audio information about whether the shutter 581 is open or closed.

The hair dryer according to a fourth embodiment of the present invention may further comprise a filter 610′. The filter 610′ may be installed on the air inlet side of the housing 510 to prevent debris contained in the air that is drawn in with the external air from being directed onto the user's hair. The hair dryer according to the present invention may further comprise an ionizer (not shown) that generates negative ions.

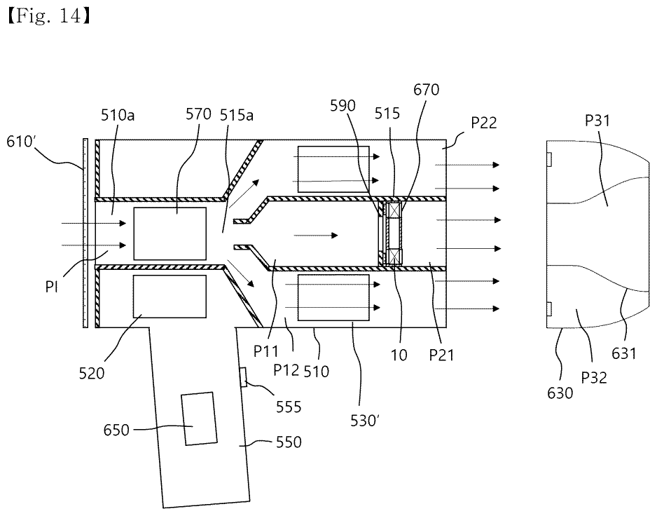

FIG. 14 is a schematic cross-sectional view of a hair dryer according to a fifth embodiment of the present invention.

Referring to FIG. 14, a hair dryer according to a fifth embodiment of the present invention may include a housing 510, a heating element 530′, a handle 550, a blowing unit 570, and a scent emitter housing 570. Compared to the hair dryer according to the third and fourth embodiments of the present invention, the partition wall 515 structure is substantially the same as the partition wall structure according to the third embodiment, and the mounting locations of the heating element 530′ and filter 610′ are substantially the same as the components of the same name according to the fourth embodiment, and therefore will not be described in detail. The present embodiment is distinguished in that it comprises a scent emitter housing 670 in a configuration that is retractable with or without the use of a scent emitter 10 installed within the partition wall 515.

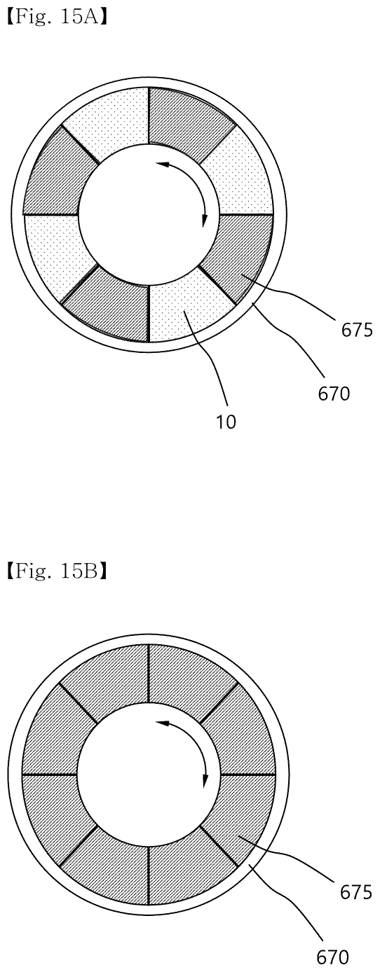

Referring to FIGS. 14, 15A, and 15B, the scent emitter housing 670, which houses the scent emitter 10, can include a shutter 675 that is retractable to remain closed when not in use and open when in use. FIG. 15a illustrates the shutter 675 in an open state, and FIG. 15b illustrates the shutter 675 in a closed state. The shutter 675 may be pivotally mounted to both sides of the scent emitter housing 670 to allow reciprocating rotational movement. The shutter 675 may be operated by a manual action of the user, or it may be operated by a driving force of a motor. Furthermore, while the shutter 675 is shown in this embodiment as opening and closing by reciprocating rotation, it can be changed to various types such as sliding movement, opening and closing movement in the blowing direction, etc.

As described above, one of ordinary skill in the art will understand that the invention may be embodied in other specific forms without departing from its technical or essential features. Therefore, the embodiments described above are to be understood as exemplary in all respects and not limiting. The scope of the invention is indicated by the following claims rather than by the detailed description, and all modifications or variations that fall within the meaning and range of equivalency of the claims are to be embraced within the scope of the invention.

1. A hair dryer comprising:
a housing having an inlet path through which external air is introduced, first and second blowing paths for blowing the air introduced from the inlet path, and an air outlet for discharging the external air;
a heating element disposed in at least one of the first and second blowing paths, the heating element configured to heat air passing therethrough;
a handle disposed on an exterior of the housing;
a blowing unit disposed in at least one of the inlet path of the housing and an interior of the handle, the blowing unit configured to draw in external air and inject the air into at least one of the first and second blowing paths; and
a scent mounting portion disposed in the housing and in communication with at least one of the first and second blowing paths, the scent mounting portion configured to removably receive a scent emitter,
wherein scent emitted from the scent emitter is discharged while bypassing the heating element.

2. The hair dryer of claim 1, further comprising a filter disposed in the housing and configured to prevent debris from entering the housing.

3. The hair dryer of claim 1,
wherein the housing further comprises a connecting passage connecting the inlet path and the second blowing path,
wherein the external air flows through the connecting passage in a direction opposite to a direction of air flow through the inlet path, and
wherein the housing further comprises:
a first partition wall separating the inlet path from the air outlet;
a wind shield configured to divert air flowing through the inlet path into the first blowing path and the connecting passage;
a second partition wall separating the first blowing path from the second blowing path; and
a third partition wall separating the connecting passage from the second blowing path, wherein an opening between the connecting passage and the second blowing path is formed at a first side of the third partition wall.

4. The hair dryer of claim 3,
wherein the scent mounting portion includes an openable/closable portion configured to open when the scent emitter is mounted and to close when the scent emitter is removed.

5. The hair dryer of claim 4,
wherein the openable/closable portion is opened and closed by:
a resilient member disposed on a support step having an expanded inner diameter at a rear portion of the third partition wall; and
a sliding member slidably disposed and actuated by a force of the scent emitter and a restoring force of the resilient member.

6. The hair dryer of claim 5,
further comprising a pressing pin formed on a side of the sliding member facing the scent emitter, the pressing pin configured to extend into the scent mounting,
wherein, when the scent emitter is mounted to the scent mounting, the pressing pin and the sliding member are pushed, and the resilient member is compressed to open the openable/closable portion,
and wherein, when the scent emitter is removed, the sliding member is retracted by a restoring force of the resilient member to close the openable/closable portion.

7. The hair dryer of claim 1,
wherein the air outlet includes a first air outlet and a second air outlet for respectively discharging air blown through the first and second blowing paths,
wherein the heating element is disposed in the first blowing path, and
wherein the scent mounting portion is disposed in the second blowing path.

8. The hair dryer of claim 7,
wherein the second blowing path is configured to wrap around an outer side of the first blowing path to prevent external surfaces of the housing from being heated by air heated by the heating element.

9. The hair dryer of claim 7,
wherein the housing has a cylindrical shape with an opening that narrows toward a first end facing the blowing unit,
and further comprises a partition wall having an opening formed at a center of the first end, and
a shutter configured to open and close the first blowing path in response to user operation.

10. The hair dryer of claim 7,
further comprising a scent emitter housing configured to receive the scent emitter, the scent emitter housing comprising a shutter that opens and closes in response to user operation.

11. The hair dryer of claim 10,
further comprising an alarm unit configured to alert a user when the shutter is opened or closed.

12. The hair dryer of claim 1,
wherein the housing further comprises a seating portion formed with an insertion hole extending through the housing,
and wherein the scent mounting portion is removably mounted to the seating portion.

13. The hair dryer of claim 11,
wherein the scent mounting portion further comprises a magnet configured to hold the scent emitter in place by magnetic force.

14. The hair dryer of claim 12,
wherein the scent mounting portion comprises:
a mounting body including a guide portion that extends through the insertion hole and is positioned in the second blowing path, the guide portion being configured to guide the emitted scent into the second blowing path, and a working space formed on one side of the guide portion;
an elastic member disposed in the working space;
a sliding member slidably disposed in the working space and configured to move in response to a force of the scent emitter and the elastic member; and
a mounting cover disposed on one side of the housing to cover the sliding member, the mounting cover having a working hole through which a portion of the sliding member protrudes, wherein the magnet is mounted on the mounting cover.

15. The hair dryer of claim 1,
further comprising a nozzle mounted on a leading edge of the housing,
wherein the nozzle comprises first and second discharge paths separated by a dividing wall extending from a boundary between the first and second blowing paths.
